(19) United States
(12) Patent Application Publication
(10) Pub. No.: US 2005/0180926 A1
(43) Pub. Date: Aug. 18, 2005

(54) Inhalable aerosol medicament for the treatment or prevention of pain

(75) Inventors: Laurent Lecourt, Sevres (FR); Franck Lescure, Paris (FR); Marc Lemaire, Paris (FR)

Correspondence Address: YOUNG & THOMPSON, 745 SOUTH 23RD STREET, 2ND FLOOR, ARLINGTON, VA, 22202, US

(73) Assignee: AIR LIQUIDE SANTE (INTERNATIONAL), PARIS (FR)

(21) Appl. No.: 11/107,809

(22) Filed: Apr. 18, 2005

Related U.S. Application Data
(63) Continuation of application No. 09/920,806, filed on Aug. 3, 2001.

(30) Foreign Application Priority Data
Aug. 3, 2000 (FR) 0010065

(51) Int. Cl. A61L 9/04; A61K 9/14; A61K 31/60

(52) U.S. Cl. 424/46; 424/600; 514/165; 514/179; 514/282

(57) ABSTRACT

The invention concerns the use of at least one gas in combination with at least one active product for manufacturing an inhalable medicament or part of an inhalable medicament intended for the treatment or prevention of pain in humans or animals. The gas is chosen from among helim, oxygen, nitrogen, xenon, hydrogen, carbon monoxide (CO), carbon dioxide (CO₂), argon, krypton, nitrogen monoxide (NO), nitrogen protoxide (N₂O), carbonated hydrocarbons, fluorocarbons and mixtures of several of these gases. The active product is chosen from among paracetamol, acetylsalicylic acid, arylcarboxylic acid, corticosteroids, mineralosteroids, non-steoroidal anti-inflammatory drugs and their derivatives, codeine and its derivatives, morphine and morphine mimetics.

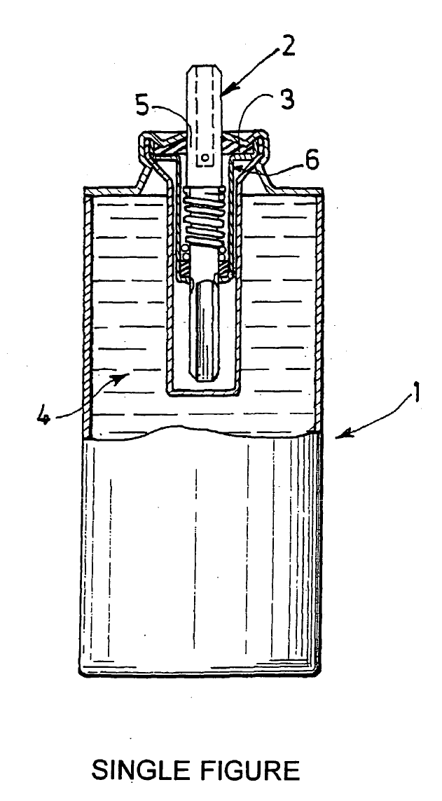

This application is a continuation of co-pending application Ser. No. 09/920,806, filed on Aug. 3, 2001, which claims the benefit of French application no. 0010065, filed on Aug. 3, 2000

The invention relates to the use of a gas or of a gas mixture, on the one hand, and of a therapeutically active product or substance, on the other hand, for manufacturing all or part of an inhalable medicament, in particular an aerosol, intended for the treatment or prevention of pain.

At present, in order to fight pain, the analgesic medicament or medicinal substance is administered either by the enteral route or by the parenteral route so that it can act and either completely or partially alleviate the feeling of pain.

The enteral route involves administering a product or an active substance via the patient's digestive tract, that is to say having the patient absorb or swallow by mouth the medicament, for example in the form of a powder, a pill, a tablet or a liquid; or introducing the active substance via the anus, for example in the form of a suppository.

However, the enteral route is more suitable for the treatment of chronic pain than for the management of acute pain.

Of the possible routes for administering medicaments aimed at fighting pain, in particular acute pain, the parenteral route is the one most used.

Administering a medicament or a therapeutically active substance to a patient by the parenteral route normally consists in injecting said medicament into said patient by means of a syringe equipped with a needle.

Injection by the parenteral route can in particular be intradermal, intravascular, intramuscular or subcutaneous, etc.

However, the problems and risks entailed in administering medicaments by the parenteral route are of several types, namely the risks to the patients, the risks to the medical staff and the efficacy/tolerance ratio of the products administered.

More precisely, as regards the risks or inconveniences to the patients, it will be readily appreciated that the puncturing of the skin caused by the injection system, in particular the needle, is often experienced as an aggressive act, in particular by children, but also by patients whose vein system is difficult to access, for example the elderly, patients suffering from serious diseases (cancer, acquired or congenital immunodeficiency, etc.) drug addicts, etc. This feeling is linked to the repeated pain created by the puncturing of the skin during injection.

Over and above this sense of aggression, the act itself is potentially dangerous. This is because the risk of vein puncture, that is to say of damaging the blood vessels, is not inconsiderable in the case of an injection of the intramuscular or intradermal type.

Another potential risk is that of the haemorrhage which can result and the formation of a subcutaneous haematoma, in particular in haemophiliac patients or those who have had treatment with anticoagulants.

In addition, some patients are described as “uninjectable” because of the state of their vein system; because of their age, in most cases children or the elderly; because of drug abuse, for example drug addicts; because of multiple punctures or injections in the context of treatment of serious conditions, for example patients treated for cancer or malignant blood disorders, obese patients, etc.

For all these patient populations, there is a risk of lymphangitis which can cause secondary infections in immunocompromised patients, particularly those affected by conditions such as HIV, cancer, blood disorders, etc. In these patients the major risk encountered is infection via catheter, which can lead to septicaemia with organ damage of the heart, liver, kidneys, lungs, etc., necessitating removal of the catheter, instigation of antibiotic therapy which is burdensome and not without toxic risks and in particular delaying the continuation of the treatment of the initial disease.

It goes without saying that all these risks multiply and increase with the chronicity of the diseases such as cancers, chronic inflammatory diseases, etc.

Moreover, there are also obvious risks to the medical staff (nurses, midwives, doctors) who must avoid any contact whatsoever with the contaminated blood of the patient when injecting the medicament.

In addition, it has been demonstrated that a medicament administered by the enteral or parenteral route is metabolized in the majority of cases, which is referred to as the hepatic first-pass effect.

To take this effect into account, it is normally necessary either to increase the initial dose of active principle in the knowledge that only a small part of said active principle will actually be effective since unmetabolized, or to use a prodrug whose metabolism will give rise to the active drug.

In all cases, however, the ratio of the active dose to the administered dose is low, which also increases the toxic risks of the medicaments by reducing the benefits/risks ratio.

In other words, the problem we are faced with is to make available a therapeutically effective composition for fighting against pain which can be administered in a way which is safe for the patient and for the medical staff, and which therefore does not present the abovementioned problems and risks.

Moreover, said therapeutically effective composition or medicament must be easy to produce at the industrial or pharmaceutical level.

The solution which the inventors of the present invention have brought to these problems is based essentially on using the inhalation route to administer the active substances to the patient, instead of the eneteral and parenteral routes normally used.

The inhalation route or aerosol therapy can in some conditions solve the problems associated with the use of the parenteral route and to a lesser extent the enteral route.

Indeed, one of the main benefits of the inhalation route is that it allows the therapeutically active drug or substance to pass directly into the arterial blood of the patient via a substantial surface area of passage and exchange, in particular the alveolar/capillary membrane of the respiratory tract, thereby preventing hepatic or renal metabolism which normally causes degradation of some of the medicament and thus necessitates increasing the administered doses in order to take account of these losses of active substance.

Administering the medicament by the inhalation route in the form of an aerosol not only makes it possible to minimize the risks which exist in administration by the parenteral route, and without losing the efficacy of the medicament, but it also makes it possible to treat certain diseases which cannot be treated by the enteral route, even though the patients affected by these diseases are part of populations for whom the parenteral route is not very suitable or cannot be used.

To date, the inhalation route has mainly been used to treat diseases or local conditions of the airways, namely pulmonary and bronchial diseases, for example asthma.

The following documents may be mentioned in this context: EP-A-680315, EP-A-655237, WO-A-98/7419, EP-A-550031, EP-A-384371, EP-A-658101, EP-A-625046, EP-A-616525, EP-A-556256, EP-A-556239, EP-A-616523, EP-A-539674, EP-A-789557, EP-A-799024, EP-A-741588, WO-A-99/53901, and WO-A-99/55319.

Now, the use of aerosol medications in the treatment of pain has never before been described or proposed, in particular for non-pulmonary and non-bronchial diseases.

The solution provided by the invention is based on the use of at least one gas in combination with at least one active product chosen from among paracetamol, acetylsalicylic acid, arylcarboxylic acid, corticosteroids, mineralosteroids, non-steroidal anti-inflammatory drugs and their derivatives, codeine and its derivatives, morphine and morphine mimetics, for manufacturing an inhalable medicament or part of an inhalable medicament intended for the treatment or prevention of pain in humans or animals.

Depending on circumstances, the use according to the invention can include one or more of the following characteristics:
the active product is chosen from among analgesics,
the active product is chosen from among compounds with an anti-inflammatory action,
the active product is chosen from among antipyretics,
the gas is chosen from among helium, oxygen, nitrogen, xenon, hydrogen, carbon monoxide (CO), carbon dioxide (CO.sub.2), argon, krypton, nitrogen monoxide (NO), nitrogen protoxide (N.sub.2O), carbonated hydrocarbons, fluorocarbons and mixtures of several of these gases,
the inhalable medicament is in the form of an aerosol comprising said gas and said active product in the form of a powder, liquid or a powder/liquid mixture,
the inhalable medicament contains a therapeutically effective quantity of active product, in which the combination of said at least one gas with said at least one active product leads to a synergistic effect,
the inhalable medicament contains at least one gas chosen from Co and NO, preferably a gas mixture containing No and CO, and at least one active product with an anti-inflammatory action,
the inhalable medicament contains at least one gas chosen from O.sub.2 and N.sub.2O, preferably a gas mixture containing O.sub.2 and N.sub.2O and at least one active product with an analgesic or morphine-like action.

The invention also concerns an inhalable medicament formed by a combination of at least one gas and at least one active product with an analgesic action, and a device for delivering an aerosol, comprising a reservoir equipped with a reservoir opening, a membrane comprising a membrane opening, said membrane being hermetically inserted into the reservoir opening, and a valve rod passing through the membrane opening and the reservoir opening and being hermetically inserted in and sliding in the membrane opening, characterized in that the reservoir contains an inhalable medicament formed by a combination of at least one gas and at least one active product with an analgesic action, in particular a gas and/or an active product as indicated above.

In the context of the present invention, the terms “active product” are used in a general sense to designate any compound, any molecule, any active principle, any substance, any composition, either organic, mineral or vegetable, or their mixtures, in any form whatsoever, namely liquid, solid, liquid/solid mixture, suspension, dilution, emulsion which have a definite therapeutic activity in the treatment of pain and whose administration to the patient by the inhalation route, via the airways of the patient, will lessen the patient's perception or sensation of pain.

However, bronchopulmonary diseases, such as asthma, are excluded from the area of protection of the present invention.

In particular, in the context of the invention, it is conceivable to have therapeutic combinations of the CO/NO and anti-inflammatory type, O.sub.2/N.sub.2O and analgesic or morphine-like type etc.

More generally, the inhalable medicament according to the invention is formed by a combination of at least one gas and at least one active product with an analgesic action, said active product being chosen from among paracetamol, acetylsalicylic acid, arylcarboxylic acid, corticosteroids, mineralosteroids, non-steroidal anti-inflammatory drugs and their derivatives, codeine and its derivatives, morphine and morphine mimetics.

In the context of the invention, the gas is chosen from among helium, oxygen, nitrogen, xenon, hydrogen, carbon dioxide, argon, krypton, carbon monoxide (CO), nitrogen monoxide (NO), nitrogen protoxide (N.sub.2O), carbonated or fluorocarbonated hydrocarbons, and mixtures of several of these gases. The carbonated or fluorocarbonated hydrocarbons which can be used are traditionally gases or gas mixtures based on heptafluoropropane, tetrafluoroethane or other similar gases; these serve chiefly to dilute the therapeutically active gases, such as N.sub.2O, NO, CO, etc., and/or to propel the aerosol medication; they have no therapeutic action to speak of.

Using the inhalation route to administer an analgesic medicament also has other advantages for the patient, namely that by avoiding a painful act the inhalation route makes it possible to reduce the risk of infection, inflammation and haemorrhage.

Moreover, the inhalation route makes it possible to reduce the administered dose of active principle, to combine different pharmaceutical forms of synergistic active principles and to verify treatment compliance by the patient, particularly if using a special aerosol therapy device such as the device marketed by Air Liquide Sante under the brand name Optineb.TM. However, other devices can be used, as is explained below.

Moreover, by administering the medicament by the inhalation route, it becomes possible or easier to treat the patient populations described as “at risk” or “difficult”, such as immunocompromised patients, that is to say patients with cancer, blood disorders or hepatitis B or C, drug addicts, patients with HIV, haemophiliacs, or patients receiving anticoagulants, or patients whose vein system is difficult to access, for example children, the elderly or the obese.

In the context of the invention, the medicament can be prepared in a stable form and packaged, for example, in pressurized containers, such as aerosol dispensers activated by finger pressure, as described in particular in the document EP-A-708805.

Depending on circumstances, the medicament can also be prepared just before it is administered to the patient, that is to say extemporaneously.

Generally, when providing aerosol therapy according to the invention, the gases which can be used can be employed in several ways, which can be combined, namely:
to effect nebulization if considering a system of pneumatic nebulization,
as a nebula vector, irrespective of the system of nebulization employed,
for the therapeutic properties of these gases on the treatment target itself or on the nebulized active principle, particularly when the active product and the gas act in synergy.

However, nebulization and vectorization of the gas sometimes come together, that is to say the same gas nebulizes the active product and then vectorizes it in the pulmonary tract of the patient.

Conversely, when it is desirable or necessary to clearly distinguish between these two functions, it is possible to use a system such as the OPTI+.TM. device marketed by Vitalaire, which can be coupled, on the one hand, to a medical ventilator to deliver a positive pressure of a respiratory gas and, on the other hand, to an oxygen nebulization system for example.

It is estimated that effective nebulization to reach the alveoli of the lungs must generate aerosol particles of about 1 to 5 .mu.m.

The physical nature of the gas is thus to be taken into consideration and it is estimated, setting aside physiological considerations, that any gas with a physical nature similar to oxygen will be a good nebulizer gas. Oxygen or gas mixtures based on oxygen are conceivable since they make it possible to use the same gas for vectorization and nebulization.

In the same way, some gases modifying the cardiopulmonary haemodynamic parameters (heart rate, blood volume, etc.) can influence the efficacy of nebulization.

Analogously, some gases with therapeutic effects can be used as vectors of the active product nebula containing the active principle or principles, in order to increase the effect of said nebulized active principles by virtue of a synergistic action of the gas and said active principles.

Thus, the combinations given in the following table can be considered in the context of the treatment of pain.

1TABLE Combinations with a potential synergistic effect GAS ACTIVE PRINCIPLES O2 Non-steroidal anti-inflammatory Xenon Steroidal anti-inflammatory NO Aspirin CO Morphine N2O Morphine mimetics H2 Codeine and derivatives Argon Paracetamol krypton Paracetamol.

The active principle can be potentiated by the nebulization gas. For example, an active principle stored in a more stable reduced form can be potentiated by means of nebulization by a gas with greater or lesser oxidizing effect, for example 02, NO, CO or CO.sub.2.

This approach has at least the advantages of better stability of the active principle released in the body and more effective targeting of the cells concerned in the treatment.

This approach is valid for any targeted treatment of pain.

By way of example, a portable manual device for delivering an aerosol according to the invention can comprise, as is shown diagrammatically in the attached figure, a reservoir 1 equipped with a reservoir opening 6, a membrane 3 comprising a membrane opening 5, said membrane being hermetically inserted into the reservoir opening 6, and a valve rod 2 passing through the membrane opening 5 and the reservoir opening 6 and being hermetically inserted in and sliding in the membrane opening 5.

The reservoir 1 contains an inhalable medicament 4 formed by a combination of at least one gas and at least one active product with an analgesic action according to the invention.

In the reservoir 1, the active product can be in powder form, liquid form, or mixtures thereof, in particular as particles dissolved or dispersed in a liquid.

The user can dispense the aerosol contained in the container 1 by exerting a pressure on the rod 2 in the direction tending to displace said rod 2 towards the inside of the container 1.

1. A method for manufacturing an inhalable medicament or part of an inhalable medicament for the treatment or prevention of pain in humans or animals comprising:
combining gaseous xenon in combination with at least one active product chosen from among paracetamol, acetylsalicylic acid, arylcarboxylic acid, corticosteroids, mineralosteroids, non-steroidal anti-inflammatory drugs and their derivatives, codeine and its derivatives, morphine and morphine mimetics.

2. A method according to claim 1, wherein the active product is chosen from among analgesics.

3. A method according to claim 1, wherein the active product is chosen from among compounds with an anti-inflammatory action.

4. A method according to claim 1, wherein the active product is chosen from among antipyretics.

5. A method according to claim 1, wherein the medicament further contains at least one additional gas chosen from among helium, oxygen, nitrogen, hydrogen, carbon monoxide (CO), carbon dioxide (CO.sub.2), argon, krypton, nitrogen monoxide (NO), nitrogen protoxide (N.sub.2O), carbonated hydrocarbons, fluorocarbons and mixtures of several of these gases.

6. A method according to claim 1, wherein the inhalable medicament is in the form of an aerosol comprising xenon and said active product in the form of a powder, liquid or a powder/liquid mixture.

7. A method according to claim 1, wherein the inhalable medicament contains a therapeutically effective quantity of active product.

8. A method according to claim 1, in which the combination of xenon with said at least one active product leads to a synergistic effect.

9. The method according to claim 1, in which the inhalable medicament contains at least one gas chosen from CO and NO, preferably a gas mixture containing NO and CO, and at least one active product with an anti-inflammatory action.

10. The method according to claim 1, in which the inhalable medicament contains at least one gas chosen from O.sub.2 and N.sub.2O.

11. The method according to claim 1, in which the inhalable medicament contains O.sub.2 and N.sub.2O, and at least one active product with an analgesic or morphine-like action.

12. Inhalable medicament formed by a combination of at least xenon and at least one active product with an analgesic action, said active product being chosen from among paracetamol, acetylsalicylic acid, arylcarboxylic acid, corticosteroids, mineralosteroids, non-steroidal anti-inflammatory drugs and their derivatives, codeine and its derivatives, morphine and morphine mimetics.

13. Medicament according to claim 12, further comprising at least one additional gas chosen from among helium, oxygen, nitrogen, hydrogen, carbon monoxide (CO), carbon dioxide (CO.sub.2), argon, krypton, nitrogen monoxide (NO), nitrogen protoxide (N.sub.2O), carbonated hydrocarbons, fluorocarbons and mixtures of several of these gases.

14. Medicament according to claim 12, characterized in that it contains a therapeutically effective amount of active product.

15. A method for treatment or prevention of pain in humans or animals comprising the step of administrating by inhalation an inhalable medicament formed by a combination of at least xenon and at least one active product with an analgesic action, said active product being chosen from among paracetamol, acetylsalicylic acid, arylcarboxylic acid, corticosteroids, mineralosteroids, non-steroidal anti-inflammatory drugs and their derivatives, codeine and its derivatives, morphine and morphine mimetics.

16. A method according to claim 15, wherein the inhalable medicament further comprises at least one additional gas chosen from among helium, oxygen, nitrogen, hydrogen, carbon monoxide (CO), carbon dioxide (CO.sub.2), argon, krypton, nitrogen monoxide (NO), nitrogen protoxide (N.sub.2O), carbonated hydrocarbons, fluorocarbons and mixtures of several of these gases.

17. Method according to claim 15, wherein the inhalable medicament contains a therapeutically effective amount of active product.

18. Method according to claim 15, wherein the active product is chosen from among analgesics.

19. Method according to claim 15, wherein the active product is chosen from among compounds with an anti-inflammatory action.

20. Method according to claim 15, wherein the active product is chosen from among antipyretics.
